(19) United States
(12) Patent Application Publication
(10) Pub. No.: US 2007/0181128 A1
(43) Pub. Date: Aug. 9, 2007

(54) Altitude simulation module II

(76) Inventors: Randolph Warren Stroetz, Rochester, MN (US); Bruce John Walters, Mazeppa, MN (US)

Correspondence Address: Randolph W. Stroetz, 5604 26th Avenue NW, Rochester, MN, 55901, US

(21) Appl. No.: 11/347,036

(22) Filed: Feb. 3, 2006

(51) Int. Cl. A61M 16/00; A62B 7/00; F16K 31/02

(52) U.S. Cl. 128/204.22; 128/204.23; 128/204.21

(57) ABSTRACT

Altitude simulation breathing systems create at near sea level, oxygen partial pressure equivalents to a desired “simulated” above ground level altitude by gas mixing and induce low oxygen content (hypoxia) in a subject through the identical physiologic mechanisms as high altitude. At the heart of all prior art is the oxygen sensor; all decisions about gas mixing are based on a direct measurement of oxygen concentration. These sensors respond slowly requiring them to be used with a reservoir; a volume of gas maintained at a given oxygen concentration. The current invention uses flow based technology and eliminates reservoir associated shortcomings. Central to the function of the current invention is the ratiometric addition in real time of nitrogen to inspired room air which is unpressurized, uncontrolled, and inspired normally. In short, we present new technology to this field not based on oxygen concentration that offers significant improvements in safety, reduced mechanical complexity, and size.

### CROSS REFERENCE: RELATED US APPLICATIONS

US patent application #US 20050202374A1 filed Jan 6, 2005 Provisional application No. 60/534,628 filed Jan. 6, 2004

### STATEMENT OF FEDERALLY SPONSORED RESEARCH/DEVELOPMENT (IF ANY)

(None)

### SEQUENCE LISTING

(None)

### BACKGROUND OF THE INVENTION

1. Field of the Invention

The present invention relates to a method and system for providing air with a lowered oxygen concentration to a human or other subject. Specifically, the invention relates to a method and system that creates at near sea level, oxygen partial pressure equivalents to a desired “simulated” above ground level altitude and creates hypoxia in a subject through the identical physiologic mechanisms as high altitude.

2. Altitude Physiology/Definition of Terms

Simulated altitude, or physiological altitude is defined as the partial pressure of oxygen that corresponds to a particular actual altitude. Upon ascention, the normal concentration of atmospheric gases does not change with altitude. However, total barometric pressure scales largely with altitude and temperature; the Babinet equation can be used to calculate the relationship between altitude, temperature and atmospheric pressure. It is shown below:
Z=C×(Bo−B)/(Bo+B)
Z=The difference in altitude in feet.
C=52494×(1+To+T−64)/900
Bo and B=Barometric pressures in inches of mercury at two altitudes.
To and T=Farenheit temperatures at the two altitudes.

A gas in a mixture exerts a “partial pressure” proportional to its' fraction of the total pressure as per Dalton's law of partial pressure. Accordingly, the partial pressure of oxygen is influenced both by its' concentration and the atmospheric pressure.

It has long been known that the partial pressure of oxygen (Po₂) is what most living organisms especially humans are sensitive to and lowering the Po₂ below a threshold value (hypoxia) will induce graded symptoms from whole organism down to cellular level. Chronic safe exposures to mild reductions in Po₂ induce physiologic mechanisms of acclimatization which are known to benefit athletes, rock climbers, and any human endeavor at high altitudes. Short term exposure at simulated high altitudes can train a subject to recognize and respond to the motor skill and cognitive degradation of hypoxia before losing consciousness.

3. Description of Prior Art

There have been various attempts at providing systems for simulating different altitudes in order to study and recognize the debilitating effects of hypoxia, as well as obtain some of the advantages of simulating different altitudes for athletic training and hypoxia symptom recognition. The relevant embodiments of these are discussed immediately below.

Wartman, Vacchiano et al U.S. Pat. No. 6,871,645 Mar. 29, 2005 describes a reduced oxygen device in which nitrogen is injected into a reservoir which communicates with outside air creating a nitrogen enriched sub-environment or volume of gas from which a person can inspire. The concentration of oxygen within this reservoir is monitored by an oxygen sensor and requires considerable volume (between 150 and 500 cubic inches) to compensate for the relatively slow response (6 seconds or slower) of the oxygen sensor.

Vacchiano et al. US patent application # 20050247311 Nov. 10, 2005 describes a reduced oxygen device which similarly blends gas in a reservoir which is monitored by an oxygen sensor. Both nitrogen and air are fed under pressure via two “off the shelf” thermal mass flow controller units, one each for air and nitrogen, and the reservoir is maintained at constant pressure of 5 PSIG, slightly more than 350 cmH₂O. According to the American Lung Association, even 45 cmH₂O can cause lung injury. Since the magnitude of this reservoir pressure (which is in direct communication with the subject during inspiration) is sufficient to cause immediate lung injury to the subject, an overpressure valve and backup system is required to reduce the possibility of overdistention lung injury. A further concern with this method of blending gas is the closed nature of the pneumatic circuit. This device is attached to the human trainee via a closed tubing and airtight mask. In the event of gas supply or flow controller failure in the off position, the #20050247311 device does not allow the subject access to ambient air.

A necessary part of any prudent system that deliberately induces hypoxia is a means of providing immediate re-oxygenation of the subject. In the case of reduced oxygen systems with a reservoir, the resultant volume of high oxygen content gas within the reservoir can pose a significant combustion safety threat. Any vessel that contains both high oxygen concentrations and electromechanical devices such as mixing fans treated with petroleum lubricants pose an explosion threat. Accordingly, it is desirable to eliminate these safety concerns.

### SUMMARY OF THE INVENTION

The present invention is referred to herein as an “Altitude Simulation Module II” (also denoted as ASMII herein) and encompasses both a method and a system for breathing at simulated altitudes. The ASMII creates desired oxygen partial pressures by the instantaneous ratiometric addition of nitrogen to spontaneous, uncontrolled inspired ambient air as a function of continuous and instantaneous measurement of inspiratory flow, not oxygen concentration, and therefore eliminates the need for a gas reservoir and associated devices. The inlet port of the ASMII is a continuously open conduit to atmosphere, without valves of any kind. These design elements improve upon prior art in terms of safety, packaged size, simplicity, and reliability.

### DESCRIPTION OF DRAWINGS

FIG. 1. Component drawing of ASMII, not to scale

FIG. 2. Legend to FIG. 1; Equation 1

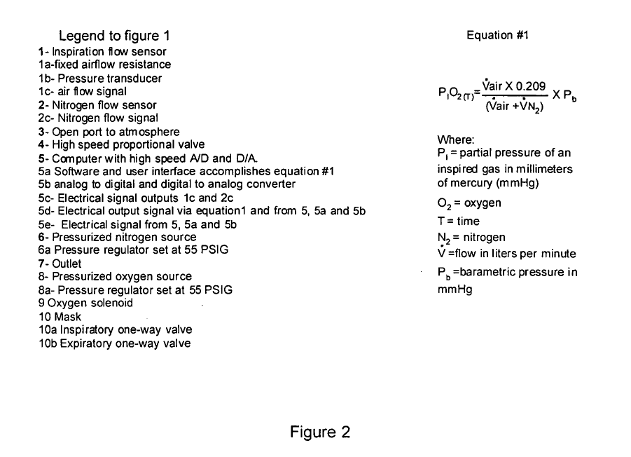

FIG. 3. Flow-time profiles of subject airflow and corresponding ratiometric nitrogen flow from ASMII

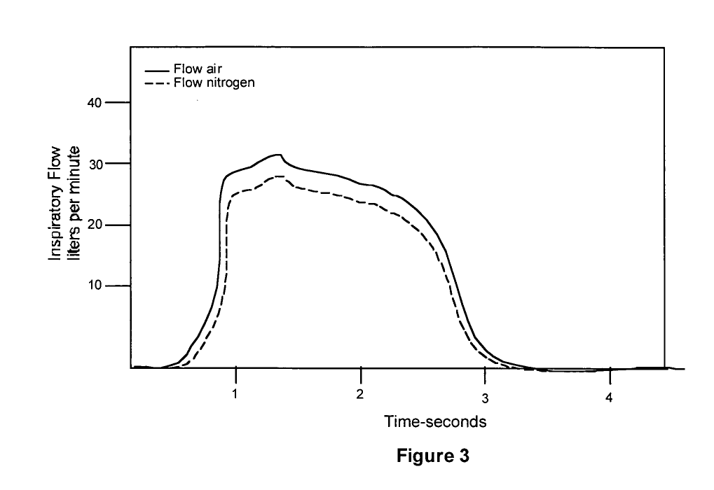

FIG. 4. Simulated altitude/time profile of a scenario

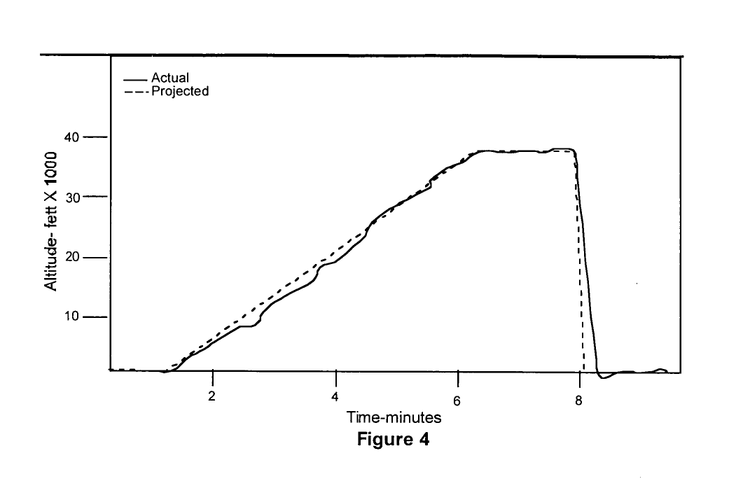

FIG. 5. Temporal and linearity response characteristics of proportional valve

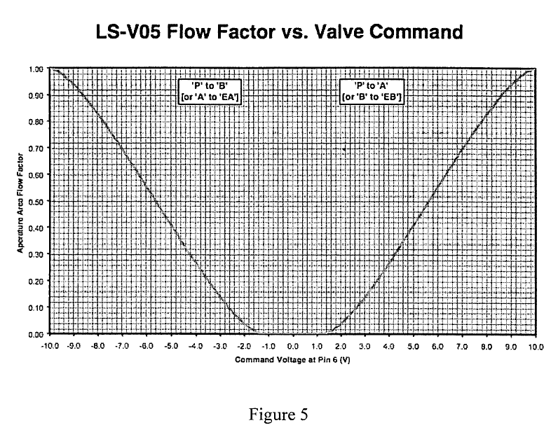

FIG. 6. Performance characteristics of flow sensors

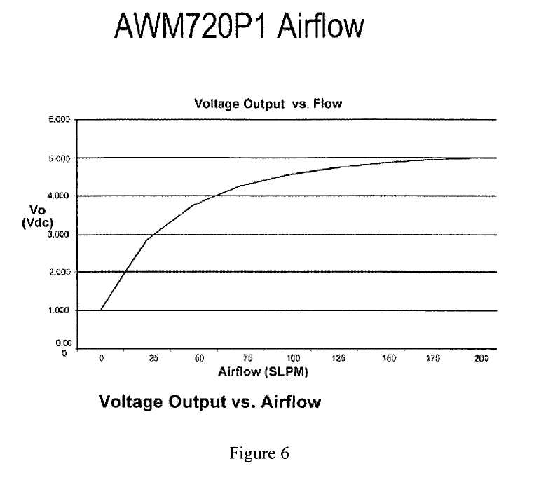

### DETAILED DESCRIPTION OF THE INVENTION

The current invention/Altitude Simulation Module II creates at near sea level, oxygen partial pressure equivalents to a desired “simulated” above ground level altitude and creates hypoxia in a subject through the identical physiologic mechanisms as high altitude. The current invention differs from prior art aimed at this goal in the following ways: While other devices claim “near instant” and “breath by breath” responses, all rely on feedback from an unheated oxygen sensor, the best of which responds in slightly more than 6 seconds. Concomitantly, the relevant prior art also rely on a volume of stored gas mixed in a reservoir or “vessel” in which the oxygen sensor resides. This prevents quick changes to the oxygen concentration within this vessel and partially compensates for the slow response of the oxygen sensor relative to within breath flow changes. During normal breathing, typical inspirations last 0.5 to 1.5 seconds, begin and end at zero flow, and typically change rapidly to 25 to 50 liters per minute or more, depending on a persons' metabolic state, size, and many other factors. Each consecutive breath can vary considerably, as does flow within a breath To adequately describe the inspired flow profile requires a sampling frequency of at least 30 Hz. Breathing systems which rely on an oxygen sensor will obviously not be able to detect within breath changes in gas concentrations let alone make appropriate corrections and are thus reliant on a reservoir to provide damping to the system as described previously. Central to the function of the current invention is the ratiometric addition in real time of nitrogen to inspired room air which is unpressurized and inspired normally. The system accomplishes oxygen partial pressure changes as a function of continuously sampled inspired flow rate. The invention does not rely on an oxygen sensor nor a reservoir as these result in temporal responses at least an order of magnitude too slow to allow near real time performance.

The ASMII adds nitrogen to inspired gas as a ratio of instantaneously measured, subject determined inspiratory flow. There is no gas reservoir or “vessel” which is kept pressurized or at a programmed oxygen level. The nitrogen is added to each breath as a ratio based on measured spontaneous flow from each breath in real-time. Since there is no reservoir to potentially become overpressurized, the need for a backpressure valve is eliminated. When oxygen is added to the system to rapidly re-oxygenate a hypoxic subject, the lack of a volume reservoir of near 100% oxygen and concomitant explosion hazard of previous altitude simulation devices is a significant improvement in safety.

The inlet port of the ASMII is a continuously open path to atmosphere, without valves of any kind. This was designed specifically to provide inherent safety as compared to prior art. Valves can fail and either block the flow of inspired air, or over distend the lungs in the case of a failed backpressure relief valve. The continuously open large bore inlet port of the ASMII is of sufficient size as to eliminate the possibility of lung overdistension even in the case of a failed nitrogen enrichment valve and provide open and easy access to ambient unpressurized room air.

### 1. DETAILED DESCRIPTION OF THE CURRENT EMBODIMENT

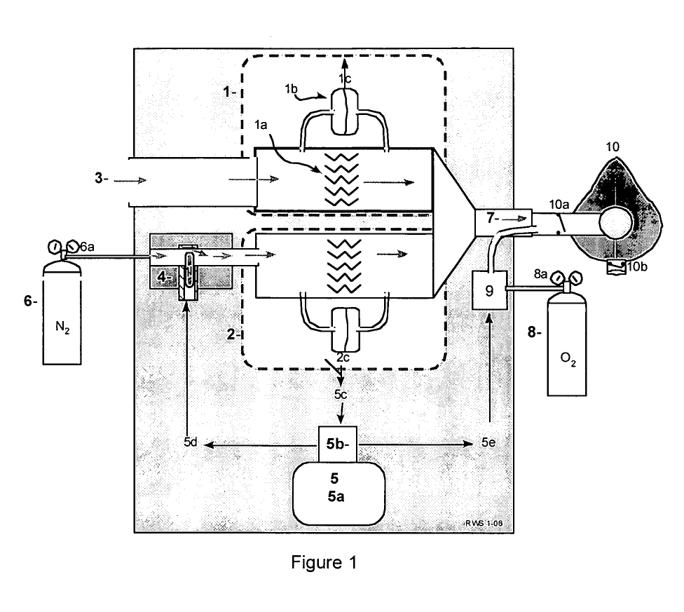

Referring to FIG. 1, the outlet port (7) is connected distally to a mask fitted with a passive one-way valve which prevents exhaled gas from entering the ASMII. As a subject inspires, air is drawn from the room through filter (3a) and inlet port (3) and through inspiratory flow sensor (1). The sensor creates a flow related change in excitation voltage as the pressure differential across resistance (1a) causes a deformation induced change in resistance within pressure transducer (1b) which is excited by voltage from an electrical power supply. This process comprises output signal (1c) which is routed to analog converter (5b) which converts the voltage based inspiratory flow signal (1c) to a 14 bit digital value. Software (5a) within computer (5) recognizes a change from the zero flow state and generates a digital command based on equation 1 to converter (5b) which converts this digital value to analog voltage signal (5d) which is routed to proportional nitrogen valve (4) and the voltage value corresponds to the degree valve (4) opens. Nitrogen flow sensor (2) is in fluid communication with nitrogen valve (4) and electrically communicates with converter (5b) hence nitrogen flow is sensed by software (5a) in an identical process as described above regarding the subjects' inspiratory flow. Corrections to nitrogen flow are continuously made as errors are sensed in an iterative process between calculated and measured nitrogen output. This is closed loop technology, more commonly known as a proportional integral derivative or PID loop. The subjects' spontaneously inspired air is combined with nitrogen in proportion to the subjects3 changing flow rate and the desired oxygen partial pressure by software (5a) according to equation 1. An oxygen sensor is in fluid communication with gas at outlet port (7) and serves as a backup monitor for device performance but is not used in the control of the gas mixing process. A pulse oximeter is connected to the subject and provides oxygen saturation and heart rate values as feedback and safety monitor. In emergency situations or where rapid re-oxygenation of the subject is desired, oxygen solenoid (9) which is in fluid communication with pressurized oxygen source (8) and outlet port (7), may be activated via user interface (5a) which activates solenoid (9) via converter (5b).

We claim:

1. An altitude simulating breathing device which operates via the following equation (equation 1)
$${P}_{I} {O}_{2 ( T )} = \frac{{V}^{•} air \times 0.209}{( {V}^{•} air + {V}^{•} {N}_{2} )} \times {P}_{b}$$
Where:
P₁=partial pressure of an inspired gas in millimeters of mercury (mmHg)
O₂=oxygen
T=time
N₂=nitrogen
\dot{V}=flow in liters per minute
P_{b}=barometric pressure in mmHg
and comprising:
a. A flow sensor #1 with a temporal response of at least 20 hertz to measure the flow of inspired air in a subject, said flow sensor in fluid communication with room air on one end and said subject on the opposite end.
b. A source of pressurized gaseous nitrogen c. A proportional valve to deliver said nitrogen, said valve having a temporal response better than 20 Hz and one end of said valve being in fluid communication with said nitrogen source.
d. A flow sensor #2 with a temporal response of at least 20 hertz with one end in fluid communication with the gas output of said nitrogen valve and the opposing end of said flow sensor #2 in fluid communication with subject end of said air flow sensor #1.
e. A computer being connected to said flow sensors and said nitrogen valve and which accomplishes said equation at the rate of at least 5 Hz.

2. The altitude simulating breathing device of claim 1 further comprising an oxygen sensor in fluid communication with gas distal to flow sensors #1 and #2 as a secondary safety monitor.

3. The breathing system of claim 1 further comprising a bacterial filter connected to room air open port of said flow sensor #1.

4. The breathing system of claim 1 further comprising a pulse oximeter connected either to said subjects' finger, earlobe, or forehead.

5. The breathing system of claim 1 further comprising the use of said flow sensor #1 to detect hyper and hypoventilation in said subject and where these conditions are further processed by software to produce a safety warning.

6. The breathing system of claim 1 further comprising a power on safety test where the functionality of all said components of claim 1 are verified. Further operation of the ASMII is prevented if there is failure of any said component.

7. The breathing system of claim 1 further comprising a pressurized oxygen source.

8. The breathing system of claim 1 further comprising an oxygen solenoid with its' inlet port in fluid communication with said oxygen source and outlet port in communication with subject end of said flow sensor #1. Oxygen solenoid is devoid of petroleum lubricants and is for emergency rapid re-oxygenation of said subject.

9. The breathing system of claim 1 further comprising a normally closed type nitrogen solenoid located between said nitrogen source and said proportional nitrogen valve. Said nitrogen solenoid is for failsafe rapid complete shutdown of nitrogen flow during electrical power failure or emergency re-oxygenation of said subject.

10. The breathing system of claim 1 further comprising a turbine in fluid communication with flow sensors 1 and 2 to provide decreased work of breathing to the subject/user.

11. The breathing system of claim 1 further comprising altitude simulating software within said computer. Said software operates said breathing device by executing equation 1 and allows programming storing and executing multiple change in simulated altitude per unit time scenarios.

12. The breathing system of claim 1 further comprising a software based operation interface via said computer.

13. The breathing system of claim 1 further comprising a network port through which access to said operation interface may be established via a remote computer.

14. The breathing system of claim 1 further comprising data conversion hardware from analog to digital and visa-versa within or connected to said computer, and which is connected electrically to all sensors and valves of claim 8.

15. The breathing system of claim 1 further comprising live audio and video capturing capability integrated with said breathing system.

16. The breathing system of claim 1 further comprising a breathing mask in fluid communication with flow sensor (a) of claim 1.

17. We claim a flow-based altitude simulating breathing device which creates desired oxygen partial pressures by the instantaneous ratiometric addition of nitrogen to uncontrolled inspired ambient air as a function of continuous and instantaneous measurement of inspiratory flow.
